(19) United States
(12) Patent Application Publication
(10) Pub. No.: US 2011/0230587 A1
(43) Pub. Date: Sep. 22, 2011

(54) FLAVORED POLYMERIC ARTICLES

(75) Inventors: Kari L. MacInnis, Copley, OH (US); Tomy Widya, Cuyahoga Falls, OH (US); Corey Capasso, Woodcliff Lake, NJ (US)

(73) Assignee: A. SCHULMAN, INC., Akron, OH (US)

(21) Appl. No.: 13/039,598

(22) Filed: Mar. 3, 2011

Related U.S. Application Data
(63) Continuation-in-part of application No. 12/583,979, filed on Aug. 28, 2009.
(60) Provisional application No. 61/190,511, filed on Aug. 29, 2008.
(60) Provisional application No. 61/310,705, filed on Mar. 4, 2010.

(30) Foreign Application Priority Data
Oct. 9, 2008 (EP) 08253299.5

(51) Int. Cl. C08L 23/06; A47J 43/27; A61K 6/00; A61K 6/10; C08L 33/10; C08L 23/12; C08K 5/053; C08K 5/1545; C08K 5/20; C08K 5/46; C08L 5/00; C08L 5/02

(52) U.S. Cl. 523/100; 523/105; 523/109; 524/560; 524/582; 524/585; 524/58; 524/387; 524/110; 524/217; 524/225; 524/56; 524/48; 524/78

(57) ABSTRACT

The present invention relates to articles comprising flavored polymeric compositions. More specifically, the invention relates to articles comprising flavored polymeric compositions comprising “flavor enhancing agents” alone, or in combination with, other flavorants. The articles comprising a flavored polymeric composition do not have to be consumed, significantly destructed, masticated or fully or partially dissolved to release flavor.

### CROSS-REFERENCE TO RELATED APPLICATIONS

The present application is a Continuation-in-Part of U.S. Ser. No. 12/583,979 filed Aug. 28, 2009, which is based on U.S. Provisional Application No. 61/190,511 filed Aug. 29, 2008 and European Application No. 08253299.5 filed Oct. 9, 2008. The present application also claims priority to U.S. Provisional Application No. 61/310,705 filed Mar. 4, 2010.

### FIELD OF THE INVENTION

Aspects of the present invention generally relate to optimization of flavored polymeric compositions and methods of producing optimized flavored polymeric compositions. More specifically, the invention relates to flavored polymeric compositions comprising “flavor enhancing agents” alone, or in combination with flavorants, wherein articles made from such polymeric compositions are not intended to be consumed, significantly destructed, masticated or fully or partially dissolved to release flavor. The polymeric compositions disclosed herein, as well as the methods for making these polymeric compositions, were developed by A. Schulman, Inc. and aspects of these inventions were previously disclosed in U.S. Ser. No. 12/583,979, U.S. Provisional Application No. 61/190,511, International Application No. PCT/US2009/004927, and European Application No. 08253299.5, all of which are expressly incorporated herein by reference.

### BACKGROUND

It is well known that flavor enhancing agents and/or flavorants are used in a wide variety of applications, such as oral rinses, toothpastes, chewing gums, dissolvable strips, tablets, beverages, confections and a variety of foodstuffs. Alone or in a variety of combinations, these flavor enhancing agents or flavorants render to the user a specific, targeted flavor sensation.

By flavorants, we refer to natural or artificial flavored substances, extracts, or essential oils. It should also be understood that in discussing flavorant or flavorants, we are referring to the use of a single flavorant or any possible combination of flavorants to achieve the desired flavor.

In reference to “flavor enhancing agents”, we refer to natural or synthetic substances which provide sweet, sour, bitter, salty, acidic, umami, fatty acid or cooling taste sensation or any combinations thereof.

In these standard applications, the flavorants, combined with other necessary ingredients, are typically distributed into the appropriate carrier for the application. Oral rinses and toothpastes would include flavorants and/or flavor enhancing agents in a liquid or paste medium. In these applications, the user experiences a rapid and appropriately intense flavor sensation due to intimate contact of the flavored ingredients in the oral cavity.

Dissolvable films and tablets also provide for intimate contact of the flavored ingredients into the oral cavity upon dissolution of the host matrix. For example, WO 02145571 teaches a fast dissolving tablet comprising a low melting point compound carrier that melts or softens at or below 37° C., a water soluble excipient, and a pharmaceutically active ingredient. Optionally, the tablet can contain flavors and flavor enhancers to deliver flavor while the tablet is dissolved. Similarly, U.S. Pat. No. 6,419,903 teaches an orally consumable film composition which is rapidly dissolvable in the oral cavity, the composition being comprised of a water soluble cellulosic carrier, a water dispersible pregelatized starch, and a flavoring agent, including sweeteners.

In the aforementioned examples, the flavorants and/or flavor enhancing agents are distributed into the appropriate carrier, such as water soluble or dissolvable carriers. The dissolution of the carrier provides effective release of the flavor ingredients into the oral cavity to achieve the targeted flavor sensation.

It is widely known that chewing and/or bubble gums also include flavorants and/or flavor enhancing agents. The gum base is a masticatory material, often water insoluble, used to carry flavors and flavor enhancing agents in gums. Gums also consist of a water soluble bulk portion. By reference, U.S. Pat. No. 5,100,678 explains that “the water-soluble portion dissipates with a portion of the flavoring agent over a period of time during chewing. The gum base portion is retained in the mouth throughout the chew.” The gum base is often comprised of natural or synthetic elastomeric materials such as chicle, natural rubber, polyvinyl acetate, styrene-butadiene copolymers, polyisobutylene, and the like. By reference, the US FDA lists approved masticatory substances for use in chewing gum under 21 CFR 172.615.

In the examples above, a commonality can be found in that these carriers provide flavor upon the consumption, mastication, dissolution, partial or full destruction of the article. Yet, there exist application needs for flavored polymeric articles which are not intended to be subjected to such end routes.

Articles produced from polymeric materials exist in a wide variety of applications. Many applications involve the polymeric article to be used or in contact with the oral cavity, but which are not intended to be masticated or dissolved. Examples of such applications include tongue depressors, mouth swabs, dental appliances, dental molds, retainers, mouthguards, protective dental films, toothbrushes, tooth bristles, dental exam gloves, pacifiers, teething toys and rings, straws, bottles, bottle caps and spouts, drink lids, utensils, adult novelty and the like. In the best case, polymeric articles used in such applications can be considered to have a neutral flavor, but more often, they are described as having a “plastic” taste which causes an unpleasant experience to the user.

As mentioned, in these other applications it is desirable to have flavored polymeric articles which can provide flavor without requiring consumption, dissolution, mastication, or destruction of the article. For these applications, flavoring an article has been achieved by either direct incorporation of the flavorant into a polymeric carrier or by applying a flavored coating to the final formed polymeric article. In the case of coated polymeric articles, the user can experience the targeted flavor sensation because the flavorant exists on the surface of the article, often in a wax carrier, to provide more intimate and immediate release to the oral cavity.

EP 0 979 208 teaches a highly flavored dental article for cleaning the interproximal surfaces of the teeth, such as dental floss comprising one filament having a flavored water-insoluble coating. The water-insoluble coating composition is comprised of a water-insoluble wax, flavor, and flavor enhancer. WO 0214448 discloses a bicomponent monofilament tape wherein the tape is made from the fusion of the sheaths of at least about 60 bicomponent coresheath fibers and capable of carrying more flavor than comparable flosses. The flavored composition is wax-based, applied as a coating, and is not integrally mixed into a polymeric base.

Drawbacks exist to this approach of applying flavored coatings on polymeric articles. One problem is that while the coating can provide the appropriate intensity and flavor, this effect is short lived because the flavorant, and even the coating itself, can be depleted from the surface and cannot be replenished. In addition, this approach causes added cost due to the secondary coating process, as well as requiring capital investment in coating machinery.

There are also problems in the case of direct incorporation of the flavorant into a polymeric carrier. A significant problem is that the intensity of flavor is weak because of the lack of sufficient flavor on the surface. Even though the flavorant may be able to migrate to the surface, especially when used in an appropriate polymeric matrix that facilitates the flavorant migration, the final effect is still greatly diminished resulting in an inferior flavor sensation. This result even extends to those articles which can be moderately compressed, e.g. by human chewing or grinding, and which still does not release adequate flavor sensation to the mouth.

In the case of articles which are not intended to be masticated, or even only mildly to moderately compressed, the use of flavored extracts alone through direct incorporation into the polymeric carrier exhibits inferior performance.

Direct incorporation of flavorants into a polymeric carrier for articles not intended to be masticated or dissolved is widely known. U.S. Pat. No. 4,971,078 describes a filter for a smoking article which includes a hollow fiber made of a thermoplastic, where a flavorant is dispersed in the thermoplastic, Such flavorants blended with thermoplastics are commercially available from such companies as International Flavors and Fragrances, Inc. WO/2008/000800 describes an oral hygiene implement made of a thermoplastic elastomer, a plasticizing oil, and a lipophilic flavoring substance. U.S. Pat. No. 6,505,961 teaches an intraoral radiographic film packet which has a thermoplastic comfort enhancing perimeter frame permanently integrated therewith during manufacture, the thermoplastic from which the comfort enhancing perimeter frame is molded having flavor/scent chemistry compounded therein prior to molding. The additives to give flavor/scent to the thermoplastic material are blended and melted in an extruder to achieve flavor/scent integral to the thermoplastic frame. US Patent Application 2007/10235039 describes a mouthguard formed of a resilient material and a flavor agent incorporated into the resilient material prior to formation of the mouthguard.

The aforementioned art relates to dispersing or embedding, i.e. direct incorporation, of flavorants into polymers which are not intended to be dissolved or masticated during use. However, there are problems with this approach. The use of flavorants alone provides predominantly a strong scent, but only affords weak taste in these instances resulting in an inferior flavor response. It is believed that such polymeric articles significantly limit the accessibility of the taste of the flavorant to the oral cavity, unlike masticatory or partially or fully dissolvable matricies, thereby causing an inferior perceived flavor. This result even extends to those polymeric articles which can be moderately compressed, e.g. by human biting, and which still does not afford adequate perception of the taste component of a flavor.

Physiologically, the sensory perception of flavor results from a combination of taste and smell. Taste is one of the traditional senses with receptors located in the taste buds found on the tongue. The four basic tastes are sweet, bitter, sour, and salty. Umami and fatty acid tastes have also been suggested as other taste categories.

Flavorants may provide both taste and aromatic components in articles which can be consumed, masticated, or significantly destructed. For flavorants in polymeric carriers which are not expected or intended to be manipulated in this way to release the flavor, the flavorant provides predominantly an aromatic component, but very little taste sensation so that the ultimate flavor perception is significantly diminished. While not wishing to be bound to a particular theory, it is believed that these types of polymers and applications do not permit enough taste sensation due to insufficient access of flavor to the oral cavity and/or the inability of the article to be manipulated (compressed) enough through the action of the user, thereby severely retarding the sensation or release of the flavor.

### SUMMARY OF THE INVENTION

Among the various aspects of the present invention is the provision of an article comprising a flavored polymeric composition. In some embodiments, the flavored article is selected from the group consisting of diet aid; pen; tongue depressor; mouth swab; dental appliance; dental mold; retainer; nightguard; mouthguard; protective dental film; toothbrush; tooth bristle; oral baby toy; pacifier; teething toy; teething ring; straw; drinking receptacle; drinking receptacle component; utensil; adult novelty device; snorkel; evacuator; ejector; aspirator; x-ray sleeve; animal trap; non-perishable animal bait; animal toy; respirator; nebulizer; peekometer; thermometer; smoking apparatus; action figure; food-like toy; artifical finger nail; cosmetic applicator; tongue depressor; shoe; shoe component; golf tee; guitar pic; wallpaper; consumer electronic device; drink stir stick; and candy stick.

In some embodiments, the flavored polymeric composition of the article comprises one or more heat stable flavor enhancing agent which provides the taste component of flavor; optionally, one or more heat stable flavorant; one or more polymer(s) characterized by a capacity for providing for flavor enhancing agent and optional flavorant migration in a finished product, as well as being characterized by thermal stability of the flavor enhancing agent and optional flavorant; and optionally, one or more additives selected from the group consisting of antioxidants, antistatics, antifogs, antimicrobials, slips, antiblocks, minerals, fillers, optical brighteners, foaming agents, nucleating agents, impact modifiers, dispersing aids, release agents, waxes, colorants, pigments, and UV stabilizers. In some configurations, the one or more polymer(s) can be selected from thermoplastics, thermosets, and silicone polymers. In some configurations, the article is not required to be consumed, significantly destructed, masticated or fully or partially dissolved to release flavor.

In some embodiments, the favored polymeric composition is formed of a masterbatch, a dry-powder or liquid concentrate. In some embodiments, the polymer is selected from polyolefins, thermoplastic polymers, thermoset polymers, and silicone polymers. In some embodiments, the polyolefin is selected form polyethylene and polypropylene, copolymers or terpolymers thereof. In some embodiments, the flavorant is selected from natural and artificial fruit, mint, and chocolate flavors. In some embodiments, the flavor enhancing agent and flavorant are suitable for human consumption. In some embodiments, the flavor enhancing agent provides the taste component of flavor. In some embodiments, the flavor enhancing agent is selected for its ability to provide sweetness, sourness, bitterness, saltiness, coolness, umami, fatty acid, or acidity. In some embodiments, the sweetness flavor enhancing agent is selected from sucrose, fructose, glucose, sorbitol, malitol, xylitol, aspartame, saccharin, sucralose, acesulfame K, mannitol, erythritol, isomalt, lactitol, maltitol, cyclamates, stevia extracts, agave nectar and the like and any combination thereof. In some embodiments, the cooling flavor enhancing agent is selected from menthol derivatives, menthyl lactate, N,2,3,-trimethyl-2-isopropylbutanamide, and the like. In some embodiments, the flavor enhancing agent providing for acidity is selected from citric acid, ascorbic acid, malic acid and fumaric acid.

In some embodiments, the compositions are compounded such that flavorant and flavor enhancing agent migrate and release or are experienced/perceived without consuming, dissolving, mastication or destruction of an article made from the composition. In some embodiments, the polymer is selected for its ability to provide flavorant and flavor enhancing agent migration such that 0-80 percent of the optional flavorant and flavor enhancing agent are retained at one hour in a use environment, 80-40 percent of the flavorant and flavor enhancing agent are retained at ten hours in a use environment and 40-20 percent of the flavorant and flavor enhancing agent are retained at one hundred hours in a use environment. In some embodiments, the flavored article is substantially free of a plastic flavor. In some embodiments, the flavored article is molded or extruded.

In some embodiments, the flavored article exhibits an optimized flavor sensation upon placement in a use environment. In some embodiments, the flavored article exhibits an optimized flavor sensation upon placement in a use environment and without consuming, dissolving, mastication or destruction of such article. In some embodiments, the flavored article exhibits enhanced flavorant and flavor enhancing agent migration such that 0-80 percent of the flavorant and flavor enhancing agent are retained at one hour in a use environment, 80-40 percent of the flavorant and flavor enhancing agent are retained at ten hours in a use environment and 40-20 percent of the flavorant and flavor enhancing agent are retained at one hundred hours in a use environment.

In some embodiments, the flavored article imparts a desirable flavor. In some embodiments, the flavored article imparts an undesirable flavor.

Another aspect provides a flavored article formed according to the following process. One or more heat stable flavorants are optionally selected. One or more heat stable flavor enhancing agents are selected. One or more polymers are selected for a capacity for providing for flavorant and flavor enhancing agent migration in a finished product, as well as thermal stability of the optional flavorant and flavor enhancing agent. The following are blended: the optional one or more flavorant(s) and one or more flavor enhancing agent(s) and one or more polymers selected from thermoplastics, thermosets, and silicone polymers, and additives selected from antioxidants, antistatics, antifogs, antimicrobials, slips, antiblocks, minerals, fillers, optical brighteners, foaming agents, nucleating agents, impact modifiers, dispersing aids, release agents, waxes, colorants, pigments, and UV stabilizers.

Other objects and features will be apparent and in part pointed out hereinafter.

### DETAILED DESCRIPTION OF THE INVENTION

It has been discovered that a polymeric article which is not intended to be consumed, significantly destructed, masticated or fully or partially dissolved to release flavor can be rendered to provide an optimized flavor by using “flavor enhancing agents” alone or, in combination with flavorants. This composition optimizes the flavor of a polymeric article by providing both the aromatic component of flavor (smell), from the use of flavorant, and the taste component of flavor, predominantly through the flavor enhancing agent. This invention also provides a long lasting effect in the article since any surface depletion of the flavorant or flavor enhancing agent is engineered to be replenished from the bulk of the polymer matrix.

The flavor enhancing agents and, optionally, the flavorants may be incorporated into the polymeric carrier by any appropriate method. An article comprising this composition may be achieved through blending flavor enhancing agents, and optionally flavorants, into polymer carriers and then subsequently melting or forming the composition into the finished article. This mixing process may be achieved by melt processing (compounding) the composition, dry mixing into a polymer powder, or mixing into a liquid polymer. The mixing process may occur at any stage of article formation. The composition may be supplied as either a masterbatch (concentrate) or fully formulated compound which may, in turn, be utilized by article manufacturers. It may also be incorporated at the finishing extruder stage of a polymer reactor.

A number of different article manufacturing processes may then utilize the optimized flavor composition, such as extrusion (film, sheet or fiber), or molding processes. The final article may be constructed as a monolayer article or may be a coextruded/multilayered article or be an overmolded composition. By such a multi-layer design, the optimized flavor composition may be incorporated into the layer which will be in contact with the oral cavity and may thereby provide further cost reduction.

Selection of the polymeric carrier is based on the desired physical properties of the final application. Common polymeric materials could be any number of thermoplastics, such as, but not limited to, polyethylene, polypropylene, any copolymers or terpolymers thereof, elastomers, thermoplastic elastomers, plastomers, ionomers, polybutadiene, polybutylene, polyvinyl chloride, polylactic acid, fluorinated polymers, polystyrenes, polyesters, and polyamides. Other polymeric materials could be used such as silicones (polymerized siloxanes), silicone rubber, rubber, latex, as well as any other thermosets or any combinations thereof. Preferably, polymers are selected which are more amorphous to enhance the migration of flavorants or flavor enhancing agents through the matrix thereby improving the sensation of the final flavor. Additionally, the polymer melting or softening point must also be considered since it relates to the temperatures which may be used in processing, or formation of the finished article. The processing temperatures preferably do not surpass the optimized thermal stability of the flavor enhancing agent or flavorants. This aspect is more important when directly incorporating the components into the polymer. Once incorporated into the polymer, through a finishing extruder, concentrate, or compound, the flavorants and flavor enhancing agents are somewhat protected by the polymeric matrix and so during subsequent processes, like molding or extrusion, may be processed at higher temperatures.

Selection of the flavorant most obviously takes into consideration the desired flavor and aroma characteristics. In addition, one must select flavorants which possess adequate thermal stability so that they will withstand the melt processing temperatures of the selected polymer without diminishing the strength or compromising the flavor. These melt processing temperatures can often range between 75° C. to 300° C. The flavorant thermal stability is dependent on the compound(s) inherent to or utilized in the production of the flavorant. In addition, some flavorants are contained in water or oil soluble carriers. In this case, oil soluble carrier systems generally provide better thermal stability versus water soluble carrier systems which are based on water, ethanol, glycol, etc. Additionally, oil soluble carriers, where present, are better suited or more compatible for the majority of polymers. Based on these criteria, thermally stable flavorants can be designed by the flavorant manufacturer using the appropriate essential oils, natural or artificial substances, or extracts, and, where needed, in an appropriate carrier to allow for incorporation into a melt processable polymer. The flavorant selected must also be one that is safe for human consumption.

Examples of fruit flavorant include flavorant imparting flavor, such as raspberry, strawberry, apple, melon, peach, etc., as well as many other fruit flavorants known in the art. Examples of mint flavorant may be selected from peppermint, cinnamon, or spearmint, as well as other mint flavorants known in the art. Examples of citrus flavorant may be selected from orange, lime or lemon, as well as ether citrus flavorants known in the art. Other flavorants such as chocolate, vanilla, etc., may be selected. The flavorant may be selected based on the desired aroma and flavor contribution, thermal stability as described previously, and its compatibility with the polymeric carrier. The flavorant may be added at parts by weight levels from 0.01 to 20%, optionally 1 to 10%, depending on the intensity of the aroma provided by the flavorant, the selected polymer, as relates to its crystallinity and corresponding effect on the regulation of the flavorant migration, as well as the thickness of the final article.

As with flavorants, the selection of flavor enhancing agents also takes into consideration the desired taste as related to the target flavor. Agents which may provide sweetness, sourness, bitterness, saltiness, coolness, umami, fatty acid or acidity may be used and depend on the final flavor target. For example, to optimize fruity or mint flavors, a fruit or mint flavorant in combination with a sweetener may be employed to provide the optimized flavor. Sweeteners can be natural or artificial of bulk or intense type, such as, but not limited to, sucrose, fructose, glucose, sorbitol, malitol, xylitol, aspartame, saccharin, sucralose, acesulfame K, mannitol, erythritol, isomalt, lactitol, maltitol, cyclamates, stevia extracts, agave nectar and the like and any combination thereof. Flavor enhancing agents may be added at levels of 0.1 to 50%, optionally at levels between 5 to 20%, by weight.

Flavor enhancing agents also need to be selected based on their thermal stability to tolerate processing temperatures, otherwise a diminished or altered taste could be experienced. The melt processing temperatures can often range between 75° C. to 300° C. By way of example, some sweeteners have poor thermal stability and during elevated temperature processing can be carmelized or burnt. Additional co-components or synergists which may protect the flavor enhancing agent during elevated temperature processing can be used to avoid change or alteration of the taste. For example, some sweeteners are combined with maltodextrin, polysaccharides, glycols, glycerides, or esters, or may be encapsulated so as to offer higher thermal stability than the neat sweetener alone. The flavor enhancing agents additionally need to be selected based on their safety for human consumption. Acidic components (acidulants) alone or in combination with flavorant and/or other flavor enhancing agents can optimize the final article flavor. Any acidic component may be considered, such as, but not limited to, citric acid, ascorbic acid, malic acid, fumaric acid, and any other organic acids or alcohols. Acidic components (acidulants) may be added at levels of 0.01 to 10%, by weight. Cooling agents (those that provide cooling sensation) may also be used alone or in combination with flavorant and/or other flavor enhancing agents to optimize flavor of the final article. Any cooling agent can be considered, such as but not limited to menthol derivatives, menthyl lactate, N,2,3,-trimethyl-2-isopropylbutanamide, and the like. Cooling agents may be added at levels of 0.05 to 20% by weight. The flavor enhancing agents additionally need to be selected based on their safety for human consumption.

The optimized flavored composition may also comprise any necessary additives which improve processibility, thermal stability, or provide performance or aesthetic attributes to the finished article. as long as these additives possess the appropriate safety status. This may include, but is not limited to, additives, such as antioxidants, antistatics, antifogs, antimicrobials, slips, antiblocks, minerals, fillers, optical brighteners, UV stabilizers, foaming agents, nucleating agents, impact modifiers, dispersing aids, release agents, waxes, colorants or pigments.

Flavor enhancing agents, flavorants, as well as additives may be compounded with many different polymers, but exhibiting the desired utility of an organoleptic perception of flavor is critical. Sub-optimal combinations are characterized by short lived organoleptic sensation, i.e., when the flavorant at the surface of the article has been expended, the organoleptic perception of flavor is lost, even though flavorant remains entrapped in the polymer of the article. Consequently, it is essential to optimize the composition to provide the desired migratory capacity of the flavor enhancing agent/flavorant/polymer combinations. Optimal compositions, when placed in a use environment, for example in a stream of flowing water, may be expected to retain approximately 82% of the perceived flavor at 1 hour, 43% at 10 hours and 38% at 100 hours. After two hours of recovery time, the flavor perception may be expected to recover to 88% of the original perceived flavor intensity in a 100 hours water-exposed specimen. It is also essential to optimize the composition to provide the desired taste component through the use of the flavor enhancing agent.

Articles

A flavored polymer including, but not limited to, flavored polymers described herein, can form of, or be incorporated into or onto, a variety of articles. For example, a flavored polymer can form of, or be incorporated into or onto, a diet aid; pen; tongue depressors or mouth swabs; dental appliances and moulds; retainers; nightguards; mouthguards; protective dental films; toothbrush and tooth bristle; pacifier, teething toy and ring; straw; drinking receptacles and components thereof; utensil; adult novelty; snorkel; evacuators, ejectors, aspirators; x-ray sleeves; rodent traps; pet toys; respirator, nebulizer; peekometer; thermometer; smoking apparatus; action figures; food-like toys; artifical or fake finger nails; cosmetic applicators (e.g., lip stick, lip gloss, chapstick applicators); tongue depressors; shoes (e.g., a flavored logo or tongue of the shoe); golf tees; guitar pics; wallpaper; consumer electronics (e.g., cellular phone); drink stirrer; candy stick; or the like.

An article comprising a flavored polymer can impart a pleasant or desirable flavor. An article comprising a flavored polymer can impart an unpleasant or undesirable flavor.

An article comprising a flavored polymer can impart a flavor to a user for at least about one hour. For example, an article comprising a flavored polymer can impart a flavor to a user for at least about two, three, four, five, six, seven, eight, nine, ten, fifteen, twenty, twenty, twenty-five, thirty, thirty-five, forty, forty-five, fifty, fifty-five, sixty, sixty-five, seventy, seventy-five, eighty, eighty-five, ninety, ninety, ninety-five, a hundred, or more hours.

Diet Aid

In some embodiments, the flavored polymer article is a diet aid device or apparatus. Flavored polymer can form all or part of the diet aid device or apparatus or be incorporated into or onto diet aid device or apparatus. For example, the flavored polymer diet aid device or apparatus can be a diet stick. A aid comprising a flavored polymer can be shaped like, for example, a candy bar, a lollipop, or a piece of gum. A diet aid comprising a flavored polymer can be of any shape compatible with the mouth of a user. For example, the diet aid comprising flavored polymer can be a rectangle, circle or other shaped piece. The diet aid comprising flavored polymer can be formed in any way known in the art, such as molded or die-cut. The diet aid comprising a flavored polymer can be bit, chewed, licked, or sucked by a user and can impart a flavor to the user. The diet aid comprising flavored polymer can impart, for example, a flavor selected from mint, lemon, cherry, orange, chocolate, as well as other flavors described herein. The diet aid comprising flavored polymer can include all FDA approved materials. The diet aid comprising a flavored polymer can be designed to not include BPA or phtalates, or other similar harmful or undesirable substances. The diet aid comprising a flavored polymer can be washable or cleanable without substantial or significant loss of flavor. A diet aid comprising a flavored polymer can impart a flavor to a user for at least about one hour. For example, a diet aid comprising a flavored polymer can impart a flavor to a user for at least about two, three, four, five, six, seven, eight, nine, ten, fifteen, twenty, twenty, twenty-five, thirty, thirty-five, forty, forty-five, fifty, fifty-five, sixty, sixty-five, seventy, seventy-five, eighty, eighty-five, ninety, ninety, ninety-five, a hundred, or more hours.

A diet aid comprising a flavored polymer can be used as a non-prescriptive or non-medicinal aid in a weight loss program. A diet aid comprising a flavored polymer can be used as a non-prescriptive or non-medicinal aid for the treatment of sugar and other foodstuff cravings experienced by dieters in the course of a diet regime. In embodiments where the diet aid comprising a flavored polymer contains no metabolizable sugars, limited metabolizable sugars, fats, sodium, or calories, the flavor experience of the diet aid can be a full or partial substitute for flavors, such as sweet flavors, derived from sugar as well as non-sugar foodstuffs. A diet aid comprising a flavored polymer can be used, for example, for stress management, a study aid, while driving, an eating substitute, during fasting periods (e.g., a religious fasting period), in a diet program, as a diet incentive, or a diet plan product accessory.

Pen

In some embodiments, the flavored polymer article is a pen. A pen is generally understood to be an implement used to apply ink or another similar substance to a surface for the purpose of writing. A flavored polymer can form all or part of the pen or be incorporated into or onto pen. In some embodiments, a pen comprising a flavored polymer can be a long, thin, rounded device. A pen comprising a flavored polymer can be, for example, a ballpoint, rollerball, fountain, or felt-tip pen. A pen comprising a flavored polymer can have a taste such that when a user places the pen in their mouth, they can taste the flavor. The pen can be formed directly of a flavored polymer. The pen can be coated with a flavored polymer. The pen can incorporate one or more components comprising a flavored polymer.

The flavored polymer pen can comprise a pen according to, for example, U.S. Pat. No. 6,899,486 (incorporated herein by reference) with a flavored polymer material as described herein.

Tongue Depressors or Mouth Swabs

In some embodiments, the flavored polymer article is a tongue depressor or mouth swab. A tongue depressor or mouth swab comprising a flavored polymer can be one piece or multiple pieces, one or more of which incorporate in whole or in part a flavored polymer. A flavored polymer can form all or part of the tongue depressor or mouth swab or be incorporated into or onto the tongue depressor or mouth swab. A tongue depressor or mouth swab comprising a flavored polymer can comprise one or more components that are placed in a subject's mouth, sometimes for extended periods. A flavored polymer can form of, or be incorporated into or onto all or part of the tongue depressor or mouth swab. For example, a flavored polymer can comprise a portion of the tongue depressor or mouth swab designed for oral contact. A tongue depressor or mouth swab comprising a flavored polymer can provide a flavor to a subject during use of such device in the mouth of a patient. A tongue depressor or mouth swab comprising a flavored polymer can be used to soothe or distract a patient during a procedure.

Dental Appliances and Molds

In some embodiments, the flavored polymer article is a dental tool, appliance, mold, or glove. A dental tool, appliance, mold, or glove comprising a flavored polymer can be one piece or multiple pieces, one or more of which incorporate in whole or in part a flavored polymer. A flavored polymer can form all or part of the dental tool, appliance, mold, or glove or be incorporated into or onto the dental tool, appliance, mold, or glove. A tool, appliance, mold, or glove comprising a flavored polymer can comprise one or more components that are placed in a subject's mouth, sometimes for extended periods. A flavored polymer can form of, or be incorporated into or onto all or part of the dental tool, appliance, mold, or glove. For example, a flavored polymer can comprise a portion of the dental tool, appliance, mold, or glove designed for oral contact. A dental tool, appliance, mold, or glove comprising a flavored polymer can provide a flavor to a subject during an oral procedure, such as a dental procedure. A dental tool, appliance, mold, or glove comprising a flavored polymer can be used to soothe or distract a patient during a procedure.

Retainers

In some embodiments, the flavored polymer article is a retainer. Retainers are generally used by a patient, during day or night, to assist in straightening of their teeth or correcting their bite. A flavored polymer can form all or part of the retainer or be incorporated into or onto the retainer. A retainer can be made of a combination of metal wiring and plastic, which can comprise a flavored polymer. A retainer comprising a flavored polymer can provide a taste, for example, a continuous taste, to the user, decrease or rid an unpleasant plastic taste, or decrease or prevent dry mouth.

Nightguard

In some embodiments, the flavored polymer article is a nightguard. A nightguard comprising a flavored polymer can be used by a subject when they sleep at night to, for example, prevent snoring or reduce or prevent TMJ. A flavored polymer can form all or part of the nightguard or be incorporated into or onto the nightguard. A nightguard can be formed of one piece comprising a flavored polymer. A nightguard can be formed of multiple pieces, one or more which can comprise a flavored polymer. A nightguard can be formed of multiple layers, one or more of which comprise a flavored polymer. A flavored polymer can be introduced into or form one or more layers of the nightguard. A nightguard comprising a flavored polymer can deliver a flavor to a person wearing the article while awake or asleep. A nightguard comprising a flavored polymer can decrease or prevent dry mouth of a user. A nightguard comprising a flavored polymer can decrease or rid an unpleasant plastic taste for the user. A nightguard comprising a flavored polymer can decrease or prevent bad breath of a user or impart fresh or fresher breath to a user.

Mouthguards

In some embodiments, the flavored polymer article is a mouthguard, mouth protector, mouth piece, or gumshield. A flavored polymer can form all or part of the mouthguard, mouth protector, mouth piece, or gumshield or be incorporated into or onto the mouthguard, mouth protector, mouth piece, or gumshield. For the purposes of discussion, features will be discussed with regard to a mouthguard but apply to a mouth protector, a mouth piece, and a gumshield as well.

A mouthguard with the flavoring technology described herein is a protective device for the mouth that covers the teeth and gums that may prevent and reduce injury to the teeth, arches, lips or gums. A flavored mouthguard described herein can be used to prevent injury in contact sports (e.g., baseball, boxing, martial arts, rugby, wrestling, football, American football, Australian football, lacrosse, basketball, figure skating, hockey, underwater hockey, water polo, skiing and snowboarding), as a therapeutic treatment (e.g., bruxism or temporomandibular joint disorder), or as part of a dental procedure (e.g., tooth bleaching, application of fluoride compositions).

In some embodiments, the flavored mouthguard comprises a thermo-plastic material manufactured that can be heated and molded (e.g., by boiling) and then placed in a subject's mouth so as to more closely conform to the subject's oral cavity, teeth, arches, lips, or gums. In some embodiments, the flavored mouthguard can be made according to an impression of a subject's teeth to provide for increased fit of the mouth protector.

In some embodiments, a mouthguard designed to align jaw position of a subject (see e.g., Makkar Pure Power Mouthguard™; Under Armour Performance Mouthwear; Bite Tech) can be flavored as described herein. In some embodiments, a mouthguard designed to optimize air flow (e.g., Under Armour O-Flow™ mouthguard) can be flavored as described herein.

In some embodiments, a flavored polymer, as described herein, can formed as, or be incorporated into or onto, a mouthguard such as any of those described in US Patent Application 2007/0151568; US Patent Application 2009/0075230; U.S. Pat. No. 4,044,762; and U.S. Pat. No. 5,339,832, each incorporated herein by reference.

In some embodiments, a flavored polymer, as described herein, can formed as, or be incorporated into or onto, a flavored mouthguard such as any of those described in US Patent Application 2010/0055233 or US Patent Application 2007/0235039, each incorporated herein by reference, where a flavored polymer described herein replaces or supplements a flavor or odorant described in the cited reference.

Toothbrush and Tooth Bristle

In some embodiments, a flavored polymer, as described herein, can formed as, or be incorporated into or onto, a toothbrush or toothbristle such as any of those described in U.S. Pat. No. 7,334,283; U.S. Pat. No. 4,408,920; and U.S. Pat. No. 3,302,230, each incorporated herein by reference.

In some embodiments, a flavored polymer, as described herein, can formed as, or be incorporated into or onto, a flavored toothbrush or toothbristle such as any of those described in US Patent Application 2009/0320226, incorporated herein by reference, where a flavored polymer described herein replaces or supplements a flavor or odorant described in the cited reference.

Pacifier, Teething Toy and Ring

In some embodiments, the flavored polymer article is an oral baby toy, such as a pacifier, soother, teether, or ring. A flavored polymer can form all or part of the oral baby toy or be incorporated into or onto the oral baby toy. An oral baby toy comprising a flavored polymer can be used orally by a child, for example, as a method of comfort or for delivery of a food or liquid. An oral baby toy can comprise a flavored polymer as described herein on the portion of the article designed for oral contact. An oral baby toy comprising a flavored polymer can provide a better experience for the baby, further soothe the baby, or aid in the delivery of a food or liquid by adding taste.

In some embodiments, a flavored polymer, as described herein, can formed as, or be incorporated into or onto an oral baby toy such as any of those described in US Patent Application 2009/0075230; U.S. Des Pat. No. D609,816; U.S. Des Pat. No. D420,447; U.S. Pat. No. 3,267,937; U.S. Pat. No. 3,669,117; and U.S. Pat. No. 4,334,382, each incorporated herein by reference.

In some embodiments, a flavored polymer, as described herein, can formed as, or be incorporated into or onto, a odorant oral baby toy such as any of those described in U.S. Pat. No. 6,482,225, incorporated herein by reference, where a flavored polymer described herein replaces or supplements a flavor or odorant described in the cited reference.

Straw

In some embodiments, the flavored polymer article is a drinking straw. A flavored polymer can form all or part of the straw or be incorporated into or onto the straw. A straw comprising a flavored polymer can be a single or multi-piece device to aid drinking of a fluid. A straw comprising a flavored polymer can be a separate article or incorporated into another article, such as a bottle. A flavored polymer can comprise all of, or a portion of (e.g., a tip) of a straw. A straw comprising a flavored polymer can impart a flavor to a fluid (e.g., water) to the user without the fluid actually having any flavor mixed into the formulation. Alternatively, a straw comprising a flavored polymer can impart an additional flavor to an already flavored fluid. When a user drinks from straw comprising a flavored polymer, a flavor can be imparted to the user from the point of oral contact (e.g., the tip of the straw) making, for example, an unflavored fluid seem flavored or an already flavored fluid seem more flavorful.

Drinking Receptacles and Components Thereof.

In some embodiments, the flavored polymer article is a drinking receptacle or component thereof. A flavored polymer can form all or part of the drinking receptacle or component thereof or be incorporated into or onto the drinking receptacle or component thereof. A drinking receptacle comprising a flavored polymer can be, for example, a single or multi-piece cup or bottle for drinking fluids. A drinking receptacle component comprising a flavored polymer can be, for example, a pop up top (e.g., on a water bottle), drinking lid (e.g., sippy or sipper cups), wand (e.g. a camelback liquid delivery system), or ring (e.g., a ring to place around the rim of a cup or glass). A drinking receptacle or component thereof comprising a flavored polymer can be a reusable receptacle or component or a single or limited use receptacle or component. A drinking receptacle or component thereof comprising a flavored polymer can impart a flavor to a fluid (e.g., water) to the user without the fluid actually having any flavor mixed into the formulation. Alternatively, a drinking receptacle or component thereof comprising a flavored polymer can impart an additional flavor to an already flavored fluid. When a user drinks from the receptacle, a flavor can be imparted to the user from the point of oral contact (e.g., water bottle tip) making, for example, an unflavored fluid seem flavored or an already flavored fluid seem more flavorful.

Utensil

In some embodiments, the flavored polymer article is a utensil, such as a fork, spoon, knife, chopstick, or stir stick. A flavored polymer can form all or part of the utensil or be incorporated into or onto the utensil. A utensil comprising a flavored polymer can impart a flavor to the user. A utensil comprising a flavored polymer can impart a flavor to the user in combination with any flavor from food being manipulated or eaten with the utensil. A utensil comprising a flavored polymer can provide, for example, a sweet, sour, or savory taste to a user. The flavor of the utensil can be the same or different as the flavor of food being manipulated or eaten with the utensil. A utensil comprising a flavored polymer can enhance a user's eating experience. A utensil comprising a flavored polymer can be used to reduce, eliminate, or mask an undesirable flavor, such as from a medicine or a liquid or food having an unpleasant or undesirable taste.

Adult Novelty

In some embodiments, the flavored polymer article is an adult novelty product or device. A flavored polymer can form all or part of the adult novelty product or device or be incorporated into or onto the adult novelty product or device. An adult novelty product or device comprising a flavored polymer can include a condom, or other sexual devices or products that can be used, for example, to enhance a sexual experience. An adult novelty product or device comprising a flavored polymer can impart a flavor to a user when the product or device, or a portion thereof, is placed in oral contact with a user. An adult novelty product or device comprising a flavored polymer can provide a flavor to a user when orally used so as to enhance a sexual experience or decrease or rid the taste of plastic or other undesirable tastes.

Snorkel

In some embodiments, the flavored polymer article is a snorkel. A snorkel is a breathing tube that allows a swimmer who is swimming along the surface of the water to breathe normally, even though the swimmer's face is just below the surface, peering down into the water. A flavored polymer can form all or part of the snorkel or be incorporated into or onto the snorkel. A snorkel comprising a flavored polymer can be a one piece snorkel or a multi-piece snorkel. The flavored polymer can form, or be incorporated into, a mouth piece of the snorkel. The flavored polymer of the snorkel can deliver a taste to the user as they have the snorkel in their mouth. In some embodiments, the snorkel can be comprised of a single piece, in which one end is put in the swimmer's mouth and the other end is designed to extend above the water. In some embodiments, the snorkel can be comprised of two or more pieces, such as a mouthpiece and a neck, which can connect to the mouthpiece and extend out of the water.

Evacuators/Ejectors/Aspirators

In some embodiments, the flavored polymer article is an evacuator, ejector, or aspirator. A flavored polymer can form all or part of the evacuator, ejector, or aspirator or be incorporated into or onto the evacuator, ejector, or aspirator. An evacuator, ejector, or aspirator can be a single or multiple part device used by, for example, a dentist or physician, for minor to major procedures. An evacuator, ejector, or aspirator can comprise a tube shaped component that is placed in a subject's mouth, sometimes for extended periods. A flavored polymer can form of, or be incorporated into or onto all or part of the evacuator, ejector, or aspirator. For example, a flavored polymer can comprise the tip of the evacuator, ejector, or aspirator designed for oral contact. An evacuator, ejector, or aspirator comprising a flavored polymer can provide a flavor to a subject during an oral procedure, such as a dental procedure. An evacuator, ejector, or aspirator comprising a flavored polymer can be used to sooth or distract a patient during a procedure.

X-Ray Sleeves

In some embodiments, the flavored polymer article is an X-ray sleeve. A flavored polymer can form all or part of the X-ray sleeve or be incorporated into or onto the X-ray sleeve. An X-ray sleeve comprising a flavored polymer can be placed into a patient's mouth for X-rays or X-ray procedures. An X-ray sleeve can be infused or coated with a flavored polymer. An X-ray sleeve comprising a flavored polymer can be used by a patient during an X-ray or related procedure so as to deliver to the user a flavor. An X-ray sleeve comprising a flavored polymer can be used to sooth or distract a patient during a procedure.

Rodent Traps

In some embodiments, the flavored polymer article is an animal trap, for example a rodent trap, or a non-perishable animal bait. Rodent traps, such as mouse or rat traps, or non-perishable animal bait can be made of or comprise plastic. A flavored polymer can form all or part of the animal trap or non-perishable bait or be incorporated into or onto the animal trap or non-perishable bait. A flavored polymer of an animal trap or non-perishable bait can attract the animal or impart a flavor when the animal bites the trap or bait. A flavored polymer of an animal trap or non-perishable bait can increase the probability of luring or catching an animal. A flavored polymer non-perishable bait can be re-used.

Pet Toys

In some embodiments, the flavored polymer article is an animal toy, such as a pet toy. A flavored polymer can form all or part of the animal toy or be incorporated into or onto the animal toy. An animal toy, such as a chew toy, comprising a flavored polymer can be in a bone or other food shape or other random or non-random shapes. An animal toy, such as a chew toy, comprising a flavored polymer can be in the form of a rope or bar or other shapes conventionally associated with animal toys. An animal toy comprising a flavored polymer can provide a more pleasurable experience for the animal user. An animal toy comprising a flavored polymer can provide increase interaction of the animal user with the toy. When the animal bites, chews, or licks an animal toy comprising a flavored polymer, a flavor can be imparted to the animal user, where such sensation can enhance the use of the toy.

In some embodiments, a flavored polymer, as described herein, can formed as, or be incorporated into or onto, an animal toy or a flavored animal toy such as any of those described in US Patent Application 2008/0141948; U.S. Pat. No. 3,871,334; U.S. Pat. No. 4,771,733; U.S. Pat. No. 4,557,219; U.S. Pat. No. 4,513,014; and US Patent Application 2006/0110501, each incorporated herein by reference, where a flavored polymer described herein replaces or supplements a flavor or odorant described in the cited reference.

Respirator, Nebulizer, or Peekometer

In some embodiments, the flavored polymer article is a respirator, nebulizer, or peekometer. A flavored polymer can form all or part of the respirator, nebulizer, or peekometer or be incorporated into or onto the respirator, nebulizer, or peekometer. Respirators, nebulizers, and peekometers, are generally understood as medical devices having a mouthpiece that connects a tube to a machine, or devices that have a mouthpiece attached directly to them. A mouthpiece, or a portion thereof, of a respirator, nebulizer, or peekometer can comprises a flavored polymer. A respirator, nebulizer, or peekometer comprising a flavored polymer in or on an oral contact portion can impart a flavor to a user. A respirator, nebulizer, or peekometer comprising a flavored polymer can be used to sooth or distract a patient during a procedure.

Thermometer

In some embodiments, the flavored polymer article is a thermometer. A flavored polymer can form all or part of the thermometer or be incorporated into or onto the thermometer. A flavored polymer can form of or be incorporated into or onto a thermometer of any shape or size. A flavored polymer can form of or be incorporated into or onto a plastic cover of a thermometer. A flavored polymer can form of or be incorporated into or onto a disposable plastic cover of a thermometer. A flavored polymer can form of or be incorporated into or onto an oral contact tip of a thermometer. A thermometer or thermometer cover comprising a flavored polymer can impart a flavor to a user during the time in which the user's temperature is being taken. A thermometer or thermometer cover comprising a flavored polymer can be used to sooth or distract a patient during a temperature taking procedure.

Smoking Apparatus

In some embodiments, the flavored polymer article is a smoking device or apparatus. A flavored polymer can form all or part of the smoking device or apparatus or be incorporated into or onto the smoking device or apparatus. A smoking device or apparatus comprising a flavored polymer can provide a sensation of smoking without actually having to smoke a substance. A smoking device or apparatus comprising a flavored polymer can enhance smoking of a substance. A smoking device or apparatus comprising a flavored polymer can be in any shape or form, but is preferably in the form of a cigarette, cigar, or tip accommodating a cigarette or cigar. A smoking device or apparatus comprising a flavored polymer can comprise, for example, a menthol or mint flavor. A smoking device or apparatus comprising a flavored polymer can impart a sensation of smoking to a user by, for example, providing jaw fixation of an oral device in combination with a flavor of, or associated with, a cigarette or cigar.

Action Figures

In some embodiments, the flavored polymer article is an action figure toy. A flavored polymer can form all or part of the action figure toy or be incorporated into or onto the action figure toy. An action figure toy comprising a flavored polymer can impart a flavor to the user on introduction to the user's mouth.

Food-like Toys

In some embodiments, the flavored polymer article is a food-like toy. A flavored polymer can form all or part of the food-like toy or be incorporated into or onto the food-like toy. Toys that are made to look like foods, such as a strawberry or apple, can comprise flavored polymer so as to impart a flavor to the user. A food-like toy comprising a flavored polymer can impart the same, substantially similar, or different taste as the food after which the toy is modeled. The flavored polymer feature of the food-like toy can provide a new function to the toy by allowing the users to put the toy in their mouth and receive an actual flavor.

Articles with Unpleasant Flavor

Any of the articles described above can comprise a flavored polymer that imparts a negative or undesirable flavor. A flavored polymer imparting a negative or undesirable flavor can form of, or be incorporated into or onto, an article that is not suitable for contact with the mouth or consumption. For example, a flavored polymer imparting a negative or undesirable flavor can form of, or be incorporated into or onto, a children's toy or component thereof small enough to be a choking hazard (e.g., Legos). As another example, a flavored polymer imparting a negative or undesirable flavor can form of, or be incorporated into or onto, a children's toy or component thereof expected to be used in an unsanitary environment. As another example, a flavored polymer imparting a negative or undesirable flavor can form of, or be incorporated into or onto, artifical or fake finger nails. As another example, a flavored polymer imparting a negative or undesirable flavor can form of, or be incorporated into or onto, a wire coating; a cap or bottle of a cleaner or other dangerous or unhealthy formulations (e.g., bleach, ammonia, lye, general purpose cleaner, etc.); an electrical outlet cover; or other materials for which it would be undesirable for a person, especially a child, to contact with their mouth. In some embodiments, a flavored polymer imparting a negative or undesirable flavor can comprise a flavorant but not an odorant.

Definitions and methods described herein are provided to better define the present invention and to guide those of ordinary skill in the art in the practice of the present invention. Unless otherwise noted, terms are to be understood according to conventional usage by those of ordinary skill in the relevant art.

In some embodiments, the numbers expressing quantities of ingredients, properties such as molecular weight, reaction conditions, and so forth, used to describe and claim certain embodiments of the invention are to be understood as being modified in some instances by the term “about.”Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Fruit Flavored Polymeric Article

A fruit flavorant, such as raspberry, strawberry, apple, melon, peach, etc. may be used at an appropriate level in a final article to achieve the right intensity of aroma. The flavorant may be selected based on the desired aroma and flavor contribution, thermal stability as described previously, and its compatibility with the polymeric carrier. The flavorant may be added at levels from 0.01 to 20%, optionally 1 to 10%, depending on the intensity of the aroma provided by the flavorant, the selected polymer, as relates to its crystallinity and corresponding effect on the regulation of the flavorant migration, as well as the thickness of the final article. This composition also comprises a flavor enhancing agent, such as a sweetener. The sweetener, such as a sucralose/maltodextrin high intensity sweetener, has improved thermal stability over neat sucralose, may be used at levels of 0.1 to 50%, optionally at levels between 5 to 20%, to provide the appropriate level of taste to the article. Loading level of the sweetener is dependent on the intensity of the sweetener, the polymer, and the thickness of the final article. The use of the flavor enhancing agent alone may be sufficient for some applications where aroma is not required.

A composition of ethylene methylacrylate (EMA), a sucraloselrnaltodextrin high intensity sweetener, KE-18822 Raspberry flavor, and vitamin E (antioxidant) is dosed into a twin screw extruder. The processing temperatures are set at 100 to 120° C. The resulting pellets are then formed into specimens through an injection molding machine at 160 to 180° C.

### Example 2

### Mint Flavored Polymeric Article

A mint flavorant, such as peppermint or spearmint, may be used at an appropriate level in a final article to achieve the right intensity of aroma. The flavorant may be selected based on the desired aroma and flavor contribution, thermal stability as described previously, and its compatibility with the polymeric carrier. The flavorant may be added at levels from 0.01 to 20%, optionally 1 to 10%, depending on the intensity of the aroma provided by the Ravorant, the selected polymer as relates to its crystallinity and corresponding effect on the regulation of the flavorant migration, as well as the thickness of the final article. This composition also comprises a flavor enhancing agent, such as a sweetener. The sweetener, such as a sucralose/maltodextrin high intensity sweetener, may be used at levels of 0.1 to 50%, optionally at levels between 5 to 20%, to provide the appropriate level of taste to the article. Loading level of the sweetener is dependent on the intensity of the sweetener, the polymer, and the thickness of the final article. The use of the flavor enhancing agent alone may be sufficient for some applications where aroma is not required.

A composition of Low Density Polyethylene (LDPE), a sucraloselmaltodextrin high intensity sweetener and KE-2 1570 Mint flavor is dosed into a twin screw extruder. The processing temperatures are set at 100 to 120° C. The resulting pellets are then formed into specimens through an injection molding machine at 160 to 180° C.

### Example 3

### Citrus Flavored Polymeric Article

A citrus flavorant, such as orange or lemon, may be used at an appropriate level in a final article to achieve the right intensity of aroma. This may be at levels from 0.01 to 20% depending en the intensity of the aroma. The flavorant may be selected based on the desired aroma and flavor contribution, thermal stability as described previously, and its compatibility with the polymeric carrier. The flavorant may be added at levels from 0.01 to 20%, optionally 1 to 10%, depending on the intensity of the aroma provided by the flavorant, the selected polymer, as relates to its crystallinity and corresponding effect on the regulation of the flavorant migration, as well as the thickness of the final article. This composition also comprises a flavor enhancing agent, such as a sweetener. The sweetener, such as a sucralose/maltodextrin high intensity sweetener, may be used at levels of 0.1 to 50%, optionally at levels between 5 to 20%, to provide the appropriate level of taste to the article. Loading level of the sweetener is dependent on the intensity of the sweetener, the polymer, and the thickness of the final article. Additionally, another flavor enhancing agent, like an acidulant, such as citric or fumaric acid, may be utilized at levels of 0.01 to 5%, depending on intensity, polymer, and thickness of the final article.

A composition of Low Density Polyethylene (LDPE), a sucraloselrnaltodextrin high intensity sweetener, Orange flavor, citric acid, and vitamin E (antioxidant) is dosed into a twin screw extruder. The processing temperatures are set at 100 to 120° C. The resulting pellets are then formed into specimens through an injection molding machine at 160 to 180° C.

### Example 4

### Thermogravimetric Analysis of Flavored Polymer

The migrating nature of flavorants in polymers is a characteristic which is critical to the utility of the instant invention. Different flavorant/polymer combinations result in different performance characteristics, an essential characteristic of which is the organoleptic perception of flavor. Many flavorants may be compounded with many different polymers, but to possess the desired utility of an organoleptic perception of flavor is critical. Sub-optimal combinations are characterized by short lived organoleptic sensation, i.e., when the flavorant at the surface of the article has been expended, the organoleptic perception of flavor is lost, even though flavorant remains entrapped in the polymer of the article. Consequently, it is essential to be able to evaluate the migratory capacity of flavorant/polymer combinations.

To this end, polymers with flavor intensities rated as 0, 60, 80, and 100% are analyzed for thermogravimetric (TGA) performance. With these data, a linear relationship between TGA measurement and percent flavor in the polymer may be obtained and plotted with the y axis representing the TGA and the x axis representing the percentage flavor in the polymer.

### Example 5

### Flavor Panel Evaluation of Polymers with Flavorants

Flavor panel evaluations are conducted on specimens molded from different flavored compositions. Each composition is first compounded using a twin screw extruder and then injection molded into bars for taste testing. Panelists are assembled to give their opinion on the taste and aroma. Each panelist is required to answer the following questions:

1. Whether the taste represents/resembles the described flavorant, i.e., fruit, mint, etc. (Yes/No).

2. Whether the loading of the flavorant is appropriate to give pleasant taste to the molded article (Rating of 0-5 with 0 means “no taste at all”, 1 is “too weak”, 3 is “just right”, and 5 is “too strong”).

3. Whether the aroma represents/resembles the described flavorant, i.e., fruit, mint, etc. (Yes/No).

4. Whether the loading of the flavorant is appropriate to give pleasant aroma to the molded article (Rating of 0-5).

Three different base resins, Low Density Polyethylene (LDPE), Linear Low Density Polyethylene (LLDPE), and Polypropylene (PP), were selected for evaluating differences in taste and aroma of the final molded articles and the migration of the flavorants. Two flavorants, raspberry and banana, were chosen for this study.

Results are summarized in Tables 1-3.

TABLE 1
Responses on whether the taste represents the described fruit,
broken down by different resin and flavorant types.

| | |
|---|---|
| Raspberry | Banana |

| Resin | Yes | No | Yes | No |
|---|---|---|---|---|
| LLDPE | 33% | 67% | 33% | 67% |
| LDPE | 44% | 56% | 39% | 61% |
| PP | 6% | 94% | 0% | 100% |
| Total | 28% | 72% | 24% | 76% |

TABLE 2
Responses on whether the aroma represents the described fruit,
broken down to different resin and flavorant types.

| | |
|---|---|
| Raspberry | Banana |

| Resin | Yes | No | Yes | No |
|---|---|---|---|---|
| LLDPE | 100% | 0% | 89% | 11% |
| LDPE | 89% | 11% | 94% | 6% |
| PP | 83% | 17% | 83% | 17% |
| Total | 91% | 9% | 89% | 11% |

TABLE 3
Overall rating on loading level, broken down
to different resin and flavorant types.

Average based on

| Category | Type | Taste | Aroma |
|---|---|---|---|
| Base resin | LLDPE | 0.74 | 2.39 |
| | LDPE | 0.76 | 2.60 |
| | PP | 0.44 | 1.49 |
| Flavor | Raspberry | 0.74 | 2.28 |
| | Banana | 0.56 | 2.04 |

The responses summarized in Tables 1 and 2 show that when using flavorant alone, aroma is much more dominant than the taste in overall flavor sensation. 91% and 89% of the respondents can smell raspberry and banana, respectively, compared to only 28% and 24% for taste. Table 3 shows that the respondents rate the aroma much closer to 3 (ideal target) than the taste. It is also observed that both aroma and taste perception in polypropylene is consistently lower than both LDPE and LLDPE, which suggests that the higher crystallinity polymers may inhibit the migration of the flavorant.

### Example 6

### Flavor Panel Evaluation of Polymers with Flavorants and Flavor Enhancing Agents

Flavor panel evaluations are conducted on molded LDPE specimens which comprise a flavorant and a flavor enhancing agent, i.e., a sweetener. Each of the compositions is first compounded using a twin screw extruder and then injection molded into bars for taste testing. Panel testing is conducted after approximately 3 weeks of conditioning the samples at room temperature to make sure the flavors do not volatilize completely within several days after the molding of the specimens. Panelists are assembled to give their opinion on the taste and aroma. Each panel taster is required to answer the following questions.

1. Whether the surface taste represents/resembles the described flavor (Yes/No).

2. Whether the formulation is appropriate to give pleasant taste to the molded article (Rating of 0-5 with 0 means “no taste at all”, 1 is “too weak”, 3 is “just right”, and 5 is “too strong”).

3. Whether the aroma represents/resembles the described flavor (Yes/No).

4. Whether the formulation is appropriate to give pleasant aroma to the molded article (Rating of 0-5).

5. Whether the flavor lasts long enough in mouth. (Yes/No).

Results are summarized in Tables 4-8.

TABLE 4
Responses on whether the taste represents the described flavor.

| Flavor | Yes | No |
|---|---|---|
| Raspberry | 93.3% | 6.7% |
| Mint | 100.0% | 0.0% |

TABLE 5
Responses on whether the aroma represents the described flavor.

| Flavor | Yes | No |
|---|---|---|
| Raspberry | 80.0% | 20.0% |
| Mint | 93.3% | 6.7% |

TABLE 6
Overall rating on raspberry flavor for three different formulations.

| Formulation | Taste | Aroma |
|---|---|---|
| 1 | 3.00 | 2.60 |
| 2 | 2.30 | 2.20 |
| 3 | 2.80 | 2.50 |
| Average | 2.70 | 2.43 |

TABLE 7
Overall rating on mint flavor for three different formulations.

| Formulation | Taste | Aroma |
|---|---|---|
| 1 | 3.30 | 3.60 |
| 2 | 2.90 | 3.00 |
| 3 | 3.10 | 3.20 |
| Average | 3.10 | 3.27 |

TABLE 8
Responses on whether the flavor lasts long enough in the mouth.

| Flavor | Yes | No |
|---|---|---|
| Raspberry | 73.3% | 26.7% |
| Mint | 93.3% | 6.7% |

The results show that both mint and raspberry specimens have good representation of the described flavors and have favorable taste and aroma (with ratings close to the ideal target of 3.0). This panel evaluation demonstrates that the use of a flavor enhancing agent, such as a sweetener, provides the taste sensation which is lacking in the earlier panel evaluations when the specimens comprised flavorant alone.

### Example 7

The objective of this example was to characterize commercially available sweeteners for their ability to be compounded into a polymer and assess the taste intensity and durability that is imparted.

Fourteen of the main commercially available natural and artificial sweeteners were selected for characterization. The reported relative intensity versus sucrose, which is used for food applications, was noted and used as a guideline to select the concentration range to be tested for each sweetener.

For each trial, the sweetener was dry-blended at the defined concentration with a 20 MI ground flake EMA polymer and then compounded on a lab scale brabender at 160° C. for 4 minutes. Upon completion of the heated mixing, the processability was assessed in terms of thermal stability, as evidenced by presence or absence of discoloration or burning, compatibility or ability to be incorporated into a polymer, along with any other observed process issues. These three facets of processability were marked as OK (acceptable) or NOK (unacceptable). A composition marked as NOK for any one of these attributes would be unable to withstand the process temperatures and/or shear forces which are present in a polymer compounding extruder and would prohibit the creation of suitable or usable material.

The resulting compounded batch was also tasted and assessed for intensity by assigning a rating of 0=no taste, 1=weak taste, 3=good taste. The taste durability was also noted as OK or NOK for compositions which had a taste intensity rating of 3. A NOK rating was assigned if the taste was depleted within 15 seconds of tasting. Any composition rated less than 3 for intensity or had durability marked as NOK would result in a material would be unable to provide the desired attributes to the polymer.

The summary of the process and taste assessments of each trial are summarized in Table 9.

Of the 14 sweeteners evaluated, only four showed evidence of favorable processability and taste intensity when compounded into a polymer. Of these, only the sucralose/maltodextrin and acesulfame K (Ace K) sweeteners had good taste intensity and durability, but it should be noted that the Ace K did leave a bitter aftertaste. The other two sweeteners (Neotame, Stevia) did not show sufficient longevity of taste when incorporated into the polymer.

TABLE 9
Characterization of processability and taste of sweeteners in polyolefins.

| | Reiative | | Processability | | | | Taste | | |
|---|---|---|---|---|---|---|---|---|---|
| | Intensity v | Wt % | Thermal | | | | Intensity | | |
| Sweetener | sucrose | Sweetener | Stability | Compatibility | Other | Observations | Rating | Durability | Comments |
| Sucrose | 1 | 20 | OK | OK | NOK | Foamy | 1 | | |
| | | 30 | NOK | OK | NOK | Discoloration, Foamy | 1 | | |
| Fructose | 1.1-1.7 | 20 | NOK | OK | | Burned on purging | 1 | | |
| | | 30 | NOK | OK | | Burned on purging | 1 | | |
| Sorbitol | 0.6 | 20 | OK | NOK | NOK | Didn't mix well, | 0 | | |
| | | | | | | Tacky | | | |
| | | 30 | OK | NOK | NOK | Didn't mix well, | 1 | | |
| | | | | | | separated out of | | | |
| | | | | | | polymer, Tacky | | | |
| Maltitol | 0.9 | 20 | NOK | OK | | Discoloration | 0 | | |
| | | 30 | OK | OK | NOK | Foamy | 1 | | |
| Xylitol | 1 | 20 | NOK | OK | NOK | Poor form for handling | 1 | | |
| | | 30 | NOK | NOK | NOK | Didn't mix well, | 1 | | |
| | | | | | | separated out of | | | |
| | | | | | | polymer, poor handling | | | |
| Mannitol | 0.6 | 20 | OK | OK | | | 0 | | |
| | | 30 | OK | OK | | | 0 | | |
| Erythritol | 0.7 | 20 | OK | OK | | | 1 | | Faint taste |
| | | | | | | | | | depletes rapidly |
| | | 30 | OK | OK | | | 1 | | Faint taste |
| | | | | | | | | | depletes rapidly |
| Aspartame | 200 | 5 | OK | OK | | | 0 | | |
| | | 10 | NOK | OK | | Discolored | 0 | | |
| Saccharin | 300-600 | 1.5 | NOK | OK | | Discolored | 0 | | |
| | | 3 | NOK | OK | | Discolored | 1 | | |
| Sucralose | 600 | 1.5 | NOK | OK | | Burnt | n/a | | |
| | | 3 | NOK | OK | | Burnt | n/a | | |
| Sucralose/ | 60 | 15 | OK | OK | | | 3 | OK | Good taste |
| maltodextrin | | | | | | | | | and longevity |
| Acesulfame | 200 | 5 | OK | OK | | | 3 | OK | Good taste |
| K | | | | | | | | | and longevity, |
| | | | | | | | | | but bitter |
| | | | | | | | | | aftertaste |
| | | 10 | OK | OK | | | 3 | NOK | Good taste |
| | | | | | | | | | intensity but |
| | | | | | | | | | no longevity |
| | | | | | | | | | and bitter |
| | | | | | | | | | aftertaste |
| Neotame | 7000-13000 | 0.2 | OK | OK | | | 1 | NOK | Taste depleted |
| | | | | | | | | | rapidly |
| | | 0.4 | OK | OK | | | 3 | | Taste depleted |
| | | | | | | | | | rapidly |
| Stevia/ | 100-300 | 10 | OK | OK | | | 1 | | Taste depleted |
| rebaudioside | | | | | | | | | rapidly |
| A | | 20 | NOK | OK | | Discolored | 3 | NOK | Taste depleted |
| | | | | | | | | | rapidly |

What is claimed is:

1. A flavored article comprising:
a favored polymeric composition comprising
one or more heat stable flavor enhancing agent which provides the taste component of flavor;
optionally, one or more heat stable flavorant;
one or more polymer(s) characterized by a capacity for providing for flavor enhancing agent and optional flavorant migration in a finished product, as well as being characterized by thermal stability of the flavor enhancing agent and optional flavorant;
optionally, one or more additives selected from the group consisting of antioxidants, antistatics, antifogs, antimicrobials, slips, antiblocks, minerals, fillers, optical brighteners, foaming agents, nucleating agents, impact modifiers, dispersing aids, release agents, waxes, colorants, pigments, and UV stabilizers;
wherein
the one or more polymer(s) are selected from thermoplastics, thermosets, and silicone polymers;
the article is not required to be consumed, significantly destructed, masticated or fully or partially dissolved to release flavor; and
the article is selected from the group consisting of diet aid; pen; tongue depressor; mouth swab; dental appliance; dental mold; retainer; nightguard; mouthguard; protective dental film; toothbrush; tooth bristle; oral baby toy; pacifier; teething toy; teething ring; straw; drinking receptacle; drinking receptacle component; utensil; adult novelty device; snorkel; evacuator; ejector; aspirator; x-ray sleeve; animal trap; non-perishable animal bait; animal toy; respirator; nebulizer; peekometer; thermometer; smoking apparatus; action figure; food-like toy; artifical finger nail; cosmetic applicator; tongue depressor; shoe; shoe component; golf tee; guitar pic; wallpaper; consumer electronic device; drink stir stick; and candy stick.

2. The flavored article of claim 1, wherein the favored polymeric composition is formed of a masterbatch, a dry-powder or liquid concentrate.

3. The flavored article of claim 1, wherein the polymer is selected from polyolefins, thermoplastic polymers, thermoset polymers, and silicone polymers.

4. The flavored article of claim 1, wherein the polyolefin is selected form polyethylene and polypropylene, copolymers or terpolymers thereof.

5. The flavored article of claim 1, wherein the flavorant is selected from natural and artificial fruit, mint, and chocolate flavors.

6. The flavored article of claim 1, wherein the flavor enhancing agent and flavorant are suitable for human consumption.

7. The flavored article of claim 1, wherein the flavor enhancing agent provides the taste component of flavor.

8. The flavored article of claim 1, wherein the flavor enhancing agent is selected for its ability to provide sweetness, sourness, bitterness, saltiness, coolness, umami, fatty acid, or acidity.

9. The flavored article of claim 8, wherein the sweetness flavor enhancing agent is selected from sucrose, fructose, glucose, sorbitol, malitol, xylitol, aspartame, saccharin, sucralose, acesulfame K, mannitol, erythritol, isomalt, lactitol, maltitol, cyclamates, stevia extracts, agave nectar and the like and any combination thereof.

10. The flavored article of claim 8, wherein the cooling flavor enhancing agent is selected from menthol derivatives, menthyl lactate, N,2,3,-trimethyl-2-isopropylbutanamide and the like.

11. The flavored article of claim 1, wherein the flavor enhancing agent providing for acidity is selected from citric acid, ascorbic acid, malic acid and fumaric acid.

12. The flavored article of claim 1, wherein the compositions are compounded such that flavor and flavor enhancing agent migrate and release or are experienced/perceived without consuming, dissolving, mastication or destruction of an article made from the composition.

13. The flavored article of claim 12, wherein the polymer is selected for its ability to provide flavorant and flavor enhancing agent migration such that 0-80 percent of the optional flavorant and flavor enhancing agent are retained at one hour in a use environment, 80-40 percent of the flavorant and flavor enhancing agent are retained at ten hours in a use environment and 40-20 percent of the flavorant and flavor enhancing agent are retained at one hundred hours in a use environment.

14. The flavored article of claim 1, which are substantially free of a plastic flavor.

15. The flavored article of claim 1, wherein the article is molded or extruded.

16. The flavored article of claim 1, which exhibits an optimized flavor sensation upon placement in a use environment.

17. The flavored article of claim 1, which exhibits an optimized flavor sensation upon placement in a use environment and without consuming, dissolving, mastication or destruction of such article.

18. The flavored article of claim 1, which exhibits enhanced flavorant and flavor enhancing agent migration such that 0-80 percent of the flavorant and flavor enhancing agent are retained at one hour in a use environment, 80-40 percent of the flavorant and flavor enhancing agent are retained at ten hours in a use environment and 40-20 percent of the flavorant and flavor enhancing agent are retained at one hundred hours in a use environment.

19. The flavored article of claim 1, wherein the article imparts a desirable flavor.

20. The flavored article of claim 1, wherein the article imparts an undesirable flavor.

21. A flavored article formed according to the process comprising:
optionally selecting one or more heat stable flavorant;
selecting one or more heat stable flavor enhancing agent;
selecting one or more polymers for a capacity for providing for flavorant and flavor enhancing agent migration in a finished product, as well as thermal stability of the optional flavorant and flavor enhancing agent; and
blending the selected optional one or more flavorant(s), one or more flavor enhancing agent(s), one or more polymers selected from the group consisting of thermoplastics, thermosets, and silicone polymers, and one or more additives selected from the group consisting of antioxidants, antistatics, antifogs, antimicrobials, slips, antiblocks, minerals, fillers, optical brighteners, foaming agents, nucleating agents, impact modifiers, dispersing aids, release agents, waxes, colorants, pigments, and UV stabilizers;
wherein the article is selected from the group consisting of diet aid; pen; tongue depressor; mouth swab; dental appliance; dental mold; retainer; nightguard; mouthguard; protective dental film; toothbrush; tooth bristle; oral baby toy; pacifier; teething toy; teething ring; straw; drinking receptacle; drinking receptacle component; utensil; adult novelty device; snorkel; evacuator; ejector; aspirator; x-ray sleeve; animal trap; non-perishable animal bait; animal toy; respirator; nebulizer; peekometer; thermometer; smoking apparatus; action figure; food-like toy; artifical finger nail; cosmetic applicator; tongue depressor; shoe; shoe component; golf tee; guitar pic; wallpaper; consumer electronic device; drink stir stick; and candy stick.
